(19) United States
(12) Patent Application Publication
(10) Pub. No.: US 2009/0015819 A1
(43) Pub. Date: Jan. 15, 2009

(54) OPTICAL ANALYSIS SYSTEM, BLOOD ANALYSIS SYSTEM AND METHOD OF DETERMINING AN AMPLITUDE OF A PRINCIPAL COMPONENT

(75) Inventors: Michael Cornelis Van Beek, Eindhoven (NL); Bernardus Leonardus Bakker, Nijmegen (NL); Wouter Harry Jacinth Rensen, Eindhoven (NL); Frank Jeroen Pieter Schuurmans, Valkenswaard (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS NV, Eindhoven (NL)

(21) Appl. No.: 10/596,563

(22) PCT Filed: Dec. 13, 2004
(86) PCT No.: PCT/IB04/52786

§ 371 Date: Jun. 16, 2006

(30) Foreign Application Priority Data
Dec. 22, 2003 (EP) 03104904.2

(51) Int. Cl. G01N 33/48

(52) U.S. Cl. 356/39

(57) ABSTRACT

The optical analysis system (20) is arranged to determine an amplitude of a principal component of an optical signal. The optical analysis system (20) comprises a multivariate optical element (5, 6) for weighing the optical signal by a spectral weighing function and a detector (7, 8) for detecting the weighed optical signal. The optical signal comprises the principal component and a further component which was not accounted for when designing the spectral weighing function. Therefore, the detected weighed optical signal comprises a part relating to the amplitude of the principal component and a further part relating to a further amplitude of the further component. The optical analysis system (20) further comprises a modulator element (13) for modulating the detected weighed optical signal. The difference between the modulated detected weighed optical signal and the detected weighed optical signal relates to the amplitude of the principal component and thus allows for determining the amplitude of the principal component in an accurate way. The blood analysis system (40) comprises such an optical analysis system (20). The method of determining an amplitude of an principal component makes use of the optical analysis system (20).

The invention relates to an optical analysis system for determining an amplitude of a principal component of an optical signal, the optical analysis system comprising a multivariate optical element for weighing the optical signal by a spectral weighing function, and a detector for detecting the weighed optical signal.

The invention further relates to a blood analysis system comprising such an optical analysis system.

The invention further relates to a method of determining an amplitude of a principal component of an optical signal, the method comprising the steps of weighing the optical signal by a multivariate optical element having a spectral weighing function, and detecting the weighed optical signal by a detector.

U.S. Pat. No. 6,198,531 discloses an embodiment of an optical analysis system described in the opening paragraph.

The known optical analysis system is part of a spectroscopic analysis system suited for, e.g., analyzing which compounds are comprised at which concentrations in a sample. It is well known that light interacting with the sample carries away information about the compounds and their concentrations. The underlying physical processes are exploited in optical spectroscopic techniques in which light of a light source such as, e.g., a laser, a lamp or light emitting diode is directed to the sample for generating an optical signal which carries this information.

For example, light may be absorbed by the sample. Alternatively or in addition, light of a known wavelength may interact with the sample thereby generating light at a different wavelength due to, e.g., a Raman scattering process. The transmitted and/or generated light then constitutes the optical signal which may also be referred to as the spectrum. The relative intensity of the optical signal as function of the wavelength is then indicative for the compounds comprised in the sample and their concentrations.

To identify the compounds comprised in the sample and to determine their concentrations the optical signal has to be analyzed. In the known optical analysis system the optical signal is analyzed by dedicated hardware comprising an optical filter. This optical filter has a transmission which depends on the wavelength, i.e. it is designed to weigh the optical signal by a spectral weighing function which is given by the wavelength dependent transmission. The spectral weighing function is chosen such that the total intensity of the weighed optical signal, i.e. of the light transmitted by the filter, is directly proportional to the concentration of a particular compound. Such an optical filter is also referred to as a multivariate optical element. This intensity may be conveniently detected by a detector such as, e.g., a photo diode. For every compound a dedicated optical filter with a characteristic spectral weighing function is used. The optical filter may be, e.g., an interference filter having a transmission constituting the desired weighing function.

For a successful implementation of this analysis scheme it is essential to know the spectral weighing functions corresponding to the compounds of interest. The spectral weighing function may be obtained by performing a principal component analysis of a set comprising N spectra of N pure compounds of known concentration where N is an integer. Each spectrum comprises the intensity of the corresponding optical signal at M different wavelengths where M is an integer as well. Typically, M is much larger than N. Each spectrum containing M intensities at corresponding M wavelengths constitutes a M dimensional vector whose M components are these intensities. These vectors are subjected to a linear-algebraic process known as singular value decomposition (SVD) which is at the heart of principal component analysis and which is well understood in this art.

As a result of the SVD a set of N eigenvectors zₙ with n being a positive integer smaller than N+1 is obtained. The eigenvectors zₙ are linear combinations of the original N spectra and often referred to as principal component vectors or regression vectors. Note, this is true for SVD, however in general the regression vector does not have to be a linear combination of the original spectra. Typically, the principal component vectors are mutually orthogonal and determined as normalized vectors with |zₙ|=1. Using the principal component vectors zₙ, the optical signal of a sample comprising the compounds of unknown concentration may be described by the combination of the normalized principal component vectors multiplied by the appropriate scalar multipliers:

x₁z₁+x₂z₂+ . . . +xₙzₙ.

The scalar multipliers xₙ with n being a positive integer smaller than N+1 may be considered the amplitudes of the principal component vectors zₙ in a given optical signal. Each multiplier xₙ can be determined by treating the optical signal as a vector in the M dimensional wavelength space and calculating the direct product of this vector with a principal component vector zₙ. The result yields the amplitude xₙ of the optical signal in the direction of the normalized eigenvector zₙ. The amplitudes xₙ correspond to the concentrations of the N compounds.

In the known optical analysis system the calculation of the direct product between the vector representing the optical signal and the principal component vector is implemented in the hardware of the optical analysis system by means of the optical filter. The optical filter has a transmittance such that it weighs the optical signal according to the components of the principal component vector, i.e. the principal component vector constitutes the spectral weighing function. The filtered optical signal may be detected by a detector which generates a signal with an amplitude proportional to the amplitude of the principal component vector and thus to the concentration of the corresponding compound.

In a physical sense, each principal component vector is a constructed “spectrum” with a shape in a wavelength range within the optical signal. In contrast to a real spectrum, a principal component vector may comprise a positive part in a first spectral range and a negative part in a second spectral range. In this case the principal component vector has positive components for the wavelengths corresponding to the first spectral range and negative components for the wavelengths corresponding to the second spectral range.

It is a disadvantage of the known optical analysis system that the amplitude of the principal component may be determined relatively inaccurately, e.g. when the optical signal comprises a further component which is not accounted for correctly in the principal component analysis. In such a case the spectral weighing function may be determined relatively inaccurately resulting in a relatively inaccurate determination of the amplitude of the principal component. In addition to the principal component weighed by the weighing function, the multivariate optical element may a least partly transmit the further component. As a result the detected weighed optical signal may comprise a part relating to the amplitude of the principal component and a further part relating to a further amplitude of the further component, the further part causing the inaccuracy. In such a case it is common practice to repeat the principal component analysis, taking into account the further component as well. This approach is, however, relatively time and money consuming. It requires a new dedicated optical filter having the new adjusted spectral weighing function. This approach may be unrealistic in situations where the further component cannot be identified or where it is not stable in time.

It is an object of the invention to provide an optical analysis system of the kind described in the opening paragraph, which is able to determine the amplitude of the principal component relatively accurately when the optical signal comprises a further component which is not accounted for correctly in the principal component analysis, without the need for an extended principal component analysis.

According to the invention the object is realized in that the optical analysis system further comprises a modulator element for modulating the detected weighed optical signal such that a difference between the modulated detected weighed optical signal and the unmodulated detected weighed optical signal relates to the amplitude of the principal component. In this case the amplitude of the principal component may be determined at least approximately from this difference. The further component is then at least partly accounted for and removed which leads to an improved accuracy without requiring an additional extended principal component analysis.

The detected weighed optical signal may be modulated such that the principal component is reduced and the further component is mainly detected. The amplitude of the principal component may then be derived approximately from the unmodulated detected weighed optical signal minus the modulated detected weighed optical signal. Alternatively, the detected weighed optical signal may be modulated such that the further component is reduced and the principal component is mainly detected. The amplitude of the principal component may then be derived approximately from the modulated detected weighed optical signal minus the unmodulated detected weighed optical signal.

The larger the modulation depth, i.e. the more effectively the respective component is reduced compared to the other component, the more accurate the amplitude of the principal component may be determined. The modulation depth may be 50% or more such as e.g. 75% or 90%. An additional calibration may be required to quantitatively correlate the change due to the modulation to the amplitude of the principal component. Such a calibration is more simple than an extended principal component analysis. The calibration may be done using a sample with a known concentration of the analyte of interest.

The optical analysis system may further comprise a signal processor for determining the amplitude of the principal component from the difference between the modulated detected weighed optical signal and the detected weighed optical signal.

According to the invention the optical signal is not restricted to optical signals having wavelengths which are visible by the human eye. The optical signal may comprise spectral components in the ultra violet (UV) and/or in the infra red (IR) spectral. Here, the IR spectral range may comprise the near infra red (NIR) and the far infra red (FIR) which has a frequency above 1 THz, and all intermediate wavelengths as well.

According to the invention the principal component is not limited to a pure principal component. Here, a pure principal component refers to a mathematically exact eigenvector for a certain compound. A principal component may also correspond to a mixture of several compounds of known concentrations.

The further component may at least in part be due to background light detected by the optical analysis system. The background light may be caused by light scattering off optical components of the optical analysis system and/or by light originating from sources outside the optical analysis system. The background light may be changing in time such that it is not possible to account for it properly in the principal component analysis.

The principal component may relate to an electronic, vibrational and/or vibronic transition of an analyte. The further component may relate to an electronic, vibrational and/or vibronic transition of a substance other than the analyte. The principal component may relate to a Raman spectrum of an analyte. The further component may relate to a fluorescence spectrum of a substance carrying the analyte and/or of the analyte itself or to a fluorescence spectrum of a substance in between the detection volume and the detector such as human skin tissue or optical elements.

The multivariate optical element may weigh the optical signal by the spectral weighing function in transmission and/or in reflection.

The spectral weighing function may be obtained in other ways than by the principal component analysis, e.g. by any mathematical orthogonalisation procedure, any multivariate analysis method, for example partial least squares (PLS), a generic algorithm or a neural network.

The modulator element may modulate the optical signal before and/or after the weighing by the multivariate optical filter.

The modulator element may be able to modulate the detected weighed optical signal with a frequency and a phase and the signal processor may be able to determine the amplitude of the principal component from the difference between the modulated detected weighed optical signal and the detected weighed optical signal having the frequency and the phase. Such a phase-sensitive detection scheme results in a relatively high signal-to-noise ratio because it reduces noise having a different frequency and/or phase.

The optical analysis system may further comprise a light source for providing light for illuminating a sample comprising a substance having a concentration and thereby generating the principal component. The amplitude of the principal component may then relate to the concentration of the substance. The relation may be a linear relation between the amplitude and the concentration. Such an optical analysis system may be a spectroscopic analysis system.

The modulator element may be arranged to modulate a property of the light provided by the light source. The part of the detected weighed optical signal relating to the amplitude of the principal component and the further part of the detected weighed optical signal relating to a further amplitude of the further component may depend on the property the light in different ways. In this way these two parts of the detected weighed optical signal may be separated in a convenient way by modulating a property of the light provided by the light source.

The property of the light to be modulated by the light source may comprise the intensity of the light. This modulation scheme may be applied when the principal component and the further component have different dependencies on the intensity of the light. For instances the principal component may have a linear dependence on the intensity-due to e.g. linear absorption and/or spontaneous Raman scattering, and the further component may be independent from the intensity and may be due to e.g. background light. In this case, the part of the detected weighed optical signal may be determined by simply reducing the intensity of the light to zero. Alternatively, the intensity may be reduced by a certain percentage, e.g. by 50% and the change in the detected weighed optical signal may be used to calculate the amplitude of the principal component which in this example is equal to twice the change in the detected weighed optical signal.

Alternatively, the principal component may be due to a non-linear optical process such as e.g. two photon absorption, stimulated Raman scattering and/or hyper-Raman scattering, and the further component may have a linear dependence on the intensity due to e.g. linear absorption and/or spontaneous Raman scattering or it may be independent from the intensity and may be due to e.g. background light. In this case the intensity may be reduced by a certain percentage, e.g. by 90%. Because of the non-linear dependence of the principal component this component is reduced much more than the further component. At the reduced intensity the detected weighed optical signal may be hence approximately attributed to the further component. When the further component scales linearly with the intensity, the further component at 100% of the light may be calculated by multiplying the signal at in this example 90% reduction by a factor of in this example nine and subtracted from the detected weighed optical signal, e.g. by the signal processor.

In yet another alternative embodiment, the principal component may have a linear dependence on the intensity due to e.g. linear absorption and/or spontaneous Raman scattering, and the further component may be due to a non-linear optical process such as e.g. two photon absorption, stimulated Raman scattering and/or hyper-Raman scattering.

The property of the light to be modulated may comprise the polarization state of the light. The polarization state of the light may be e.g. linearly polarized along a polarization axis or it may be circularly polarized. This modulation scheme may be applied when the principal component and the further component have different dependencies on the polarization state of the light. The further component may be independent of the polarization state of the light whereas the principal component depends on the polarization state of the light. In this case the change in the detected weighed optical signal is directly related to the principal component and free from the further component. Examples of the further component may be background light, a depolarized Raman scattering signal and/or a depolarized fluorescence signal. Examples of the principal component may be a polarized Raman scattering signal and/or a polarized fluorescence signal. Other examples of the principal component may be polarized transmitted light in absorption spectroscopy, polarization spectroscopy, polarimetry.

The term “depolarized” indicates that the polarization of the optical signal generated by the light is independent of the polarization state of the light. The term “polarized” indicates that the polarization of the optical signal generated by the light depends on the polarization state of the light as described by the formula:

$$( \begin{matrix}{E}_{out , s} \\ {E}_{out , p}\end{matrix} ) = ( \begin{matrix}a & b \\ c & d\end{matrix} ) ( \begin{matrix}{E}_{in , s} \\ {E}_{in , p}\end{matrix} )$$

Here we use Jones matrices to represent the polarization. E, a, b, c and d can be complex numbers to represent elliptically polarized light. a and b may be different, and/or c and d may be different.

Instead of the light the modulator element may be arranged to modulate a property of the optical signal, i.e. the optical signal is sent to the modulator element after interaction of the light with the sample, if the sample is present. The part of the detected weighed optical signal relating to the amplitude of the principal component and the further part of the detected weighed optical signal relating to a further amplitude of the further component may depend on the property the optical signal in different ways. The property of the optical signal may comprise the polarization state of the optical signal.

The property of the light to be modulated may comprise a spectral bandwidth of the light. The bandwidth of the light may be varied from a relatively broad bandwidth to a relatively small bandwidth or vice versa. This modulation scheme may be applied when the principal component and the further component have different dependencies on the bandwidth of the light. For instance the further component may be independent of the bandwidth of the light. This is e.g. the case when the further component is independent of the light. The further component may depend on the background light. Alternatively or in addition, the further component may depend on the light but may be, within certain limits, independent of the bandwidth. For instance, the further component may be due to fluorescence which may have a broad spectrum which is rather insensitive to the bandwidth of the light inducing the fluorescence. The principal component may comprise a Raman scattering signal which may have a bandwidth which may be mainly determined by the bandwidth of the light inducing the Raman signal.

The property of the light to be modulated may comprise a wavelength of the light. The wavelength of the light may be varied from a relatively short wavelength to a relatively long wavelength or vice versa. This modulation scheme may be applied when the principal component and the further component have different dependencies on the wavelength of the light. For instance the further component may be independent of the wavelength of the light. This is e.g. the case when the further component is independent of the light. The further component may depend on the background light. Alternatively or in addition, the further component may depend on the light but may be, within certain limits, independent of the wavelength. For instance, the further component may be due to fluorescence which may have a broad spectrum which is rather insensitive to the wavelength of the light inducing the fluorescence. The principal component may comprise a Raman scattering signal. The Raman scattering signal may have a spectrum with peaks the position of which depends on the wavelength of the light inducing the Raman scattering signal. Due to the modulation of the wavelength these peaks shift. The weighing function may have a structure such that the Raman peaks of the unmodulated optical signal are weighed and detected by the multivariate optical element whereas the Raman peaks of the modulated optical signal are blocked and not detected.

The multivariate optical element may have a weighing function which is adjustable. The modulator element may be arranged to modulate the weighing function of the multivariate optical element such that the part of the detected weighed optical signal relating to the amplitude of the principal component and the further part of the detected weighed optical signal relating to a further amplitude of the further component depend on the modulated weighing function in different ways. For instance the multivariate optical element may have a spectral weighing function with relatively narrow spectral bands, e.g. corresponding to a Raman wavelength of the principal component. The spectral weighing function of the multivariate optical element may be modulated such that the relatively narrow spectral bands are shifted to a wavelength where the Raman wavelength of the principle component is no longer detected. The further component may comprise a fluorescence, spectrum which is approximately constant over the range over which the spectral bands are shifted. In this way the intensity of the fluorescence may be determined at least approximately when the spectral bands in the weighing function are shifted. The intensity of the fluorescence may then be subtracted from the weighed detected optical signal when the spectral bands are not shifted.

The multivariate optical element may comprise a dispersive element such as a grating or a prism for spectrally dispersing the optical signal, and an weighing element with adjustable segments for receiving spectral components of the spectrally dispersed optical signal and for distributing the optical signal weighed by the spectral weighing-function to the detector. The modulator element may be able to modulate the adjustable segments. Alternatively or in addition, the modulator element may modulate the dispersive element. The weighing element may comprise an array of liquid crystal cells. Each of the cells may constitute an adjustable segment. The liquid crystal cell may constitute a polarization sensitive transmission filter. The transmission may be adjusted by orienting the liquid crystal molecules, e.g. by applying a voltage across the cell.

The optical analysis system according to the invention may be part of a blood analysis system arranged to analyze a sample comprising blood. The sample may be in-vivo blood, i.e. still contained in a human or an animal, or in-vitro blood, i.e. blood extracted from a human or an animal. The analyte may comprise one or more elements selected from e.g. glucose, lactate, glycohemoglobin (HbA1c), hemoglobin, hematocrit, cholesterol (total, HDL, LDL), triglycerides, urea, albumin, creatinin, oxygenation, pH, bicarbonate, and many others. The principal component may comprise the Raman spectrum of the one or more elements. The further component may comprise the fluorescence spectrum of the medium in which the one or more elements are dissolved or contained. The medium may comprise water, human or animal skin tissue, optical elements in the light path, and/or emersion medium.

These and other aspects of the optical analysis system, the blood analysis system and the method of analyzing an optical signal according to the invention will be further elucidated and described with reference to the drawings, in which:

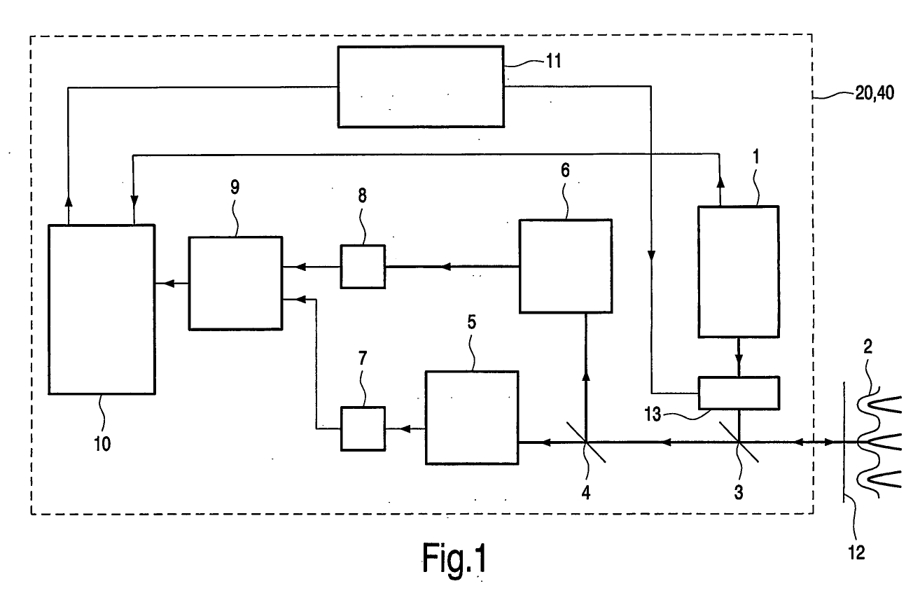

FIG. 1 is a schematic diagram of an embodiment of the optical analysis system;

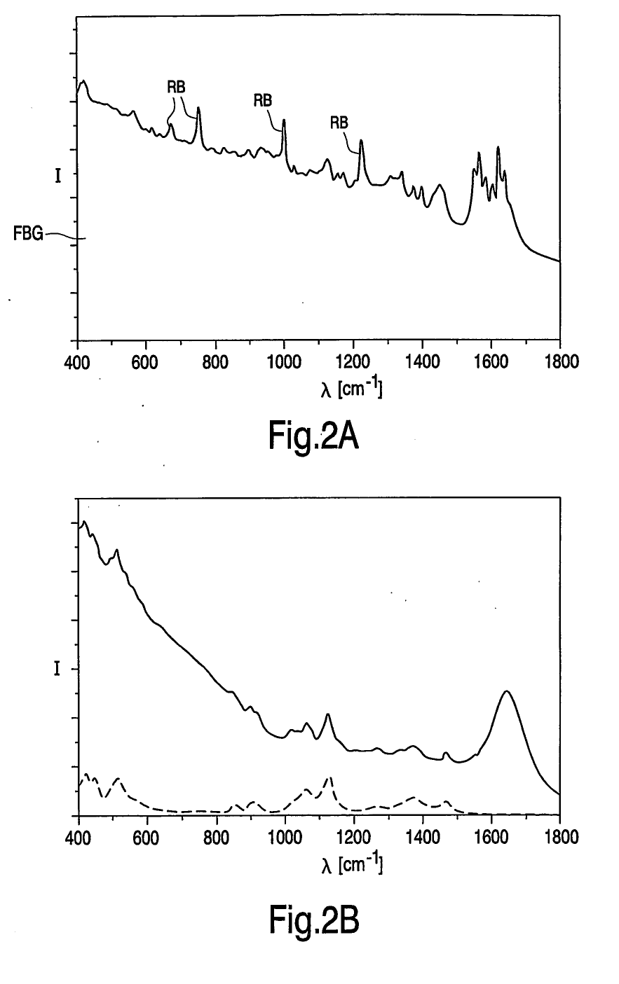

FIGS. 2A and 2B are spectra of the optical signal generated from blood in the skin and from a sample comprising one analyte in a solution;

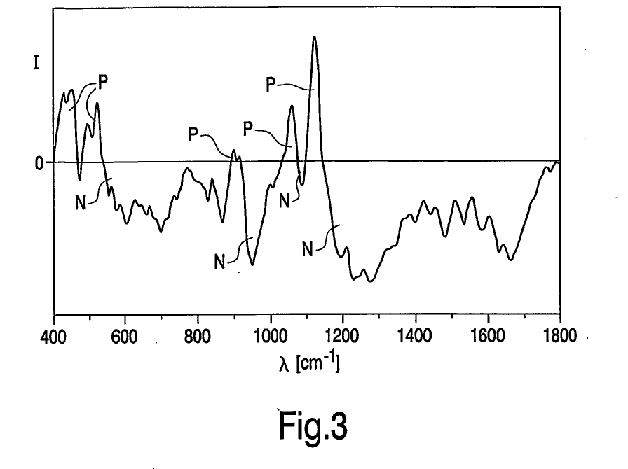

FIG. 3 is a spectral weighing function implemented in a multivariate optical element;

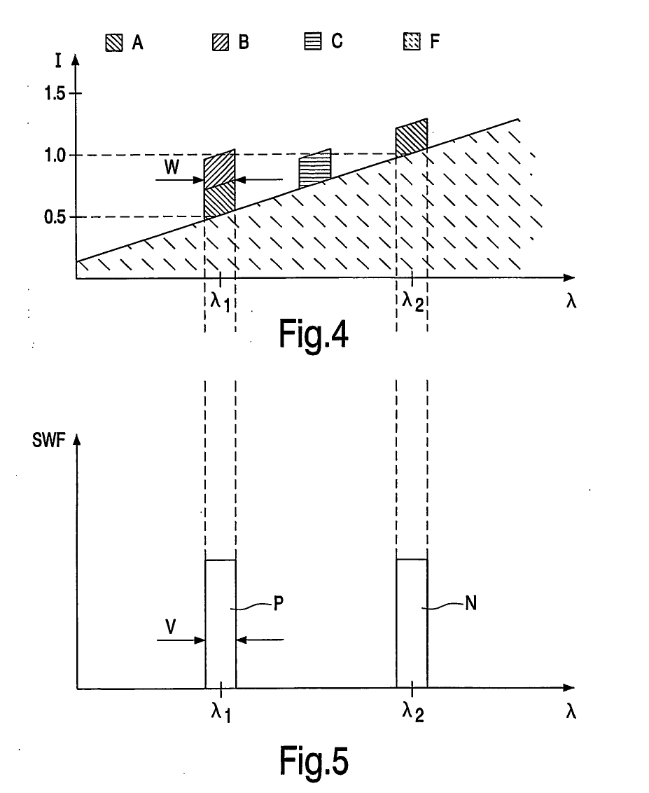

FIG. 4 is a schematic spectrum of an optical signal comprising principal components and a further component;

FIG. 5 is a schematic diagram of the spectral weighing function corresponding to one of the principal components of FIG. 4;

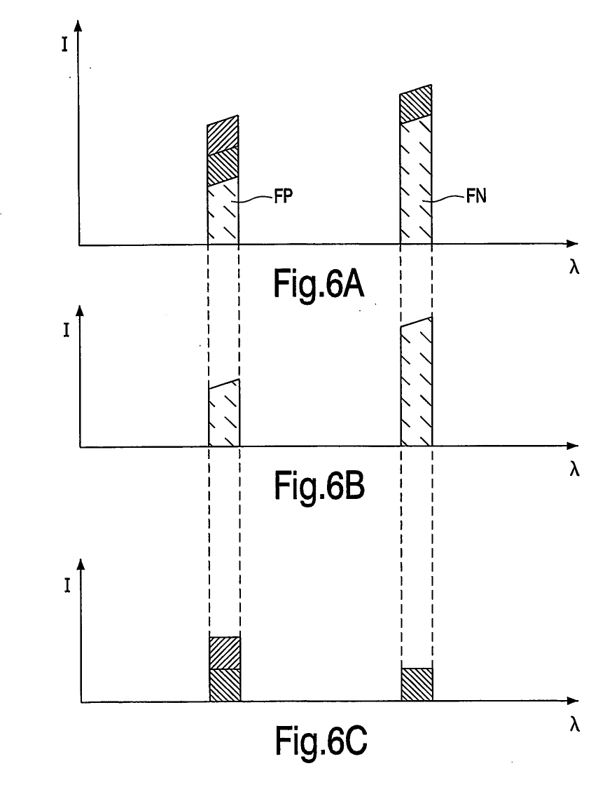

FIGS. 6A, 6B and 6C are schematic spectra of the unmodulated detected optical signal weighed by the spectral weighing functions shown in FIGS. 5A and 5B, the modulated detected optical signal weighed by the spectral weighing functions shown in FIG. 5, and the difference between these two spectra;

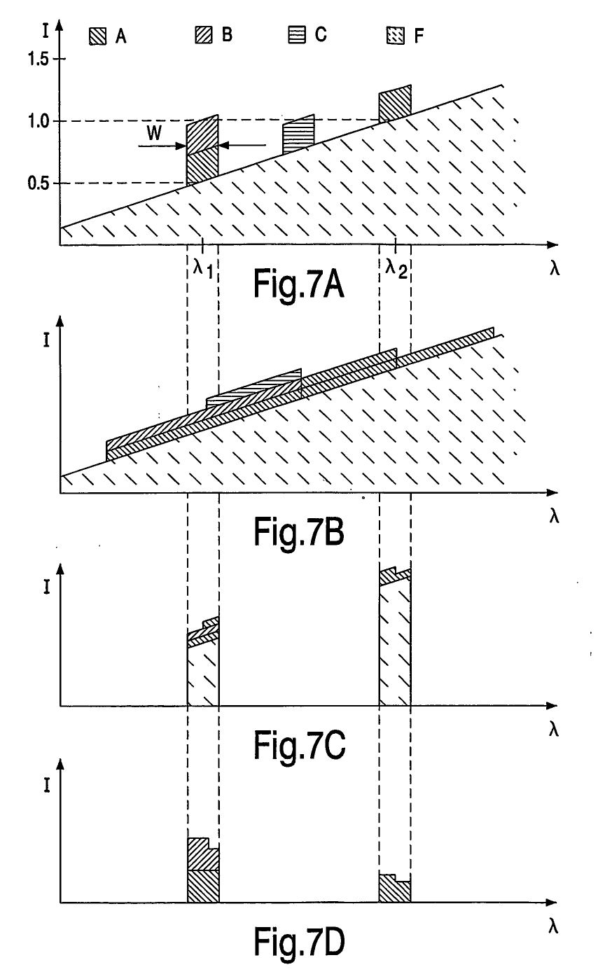

FIGS. 7A-7D are schematic spectra of the optical signal without and with modulation of the spectral bandwidth of the light provided by the light source, of the modulated detected optical signal weighed by the spectral weighing functions shown in FIG. 5, and of the difference between the unmodulated detected optical signal weighed by the spectral weighing functions shown in FIG. 5 and the modulated detected optical signal weighed by the spectral weighing functions shown in FIG. 5, respectively;

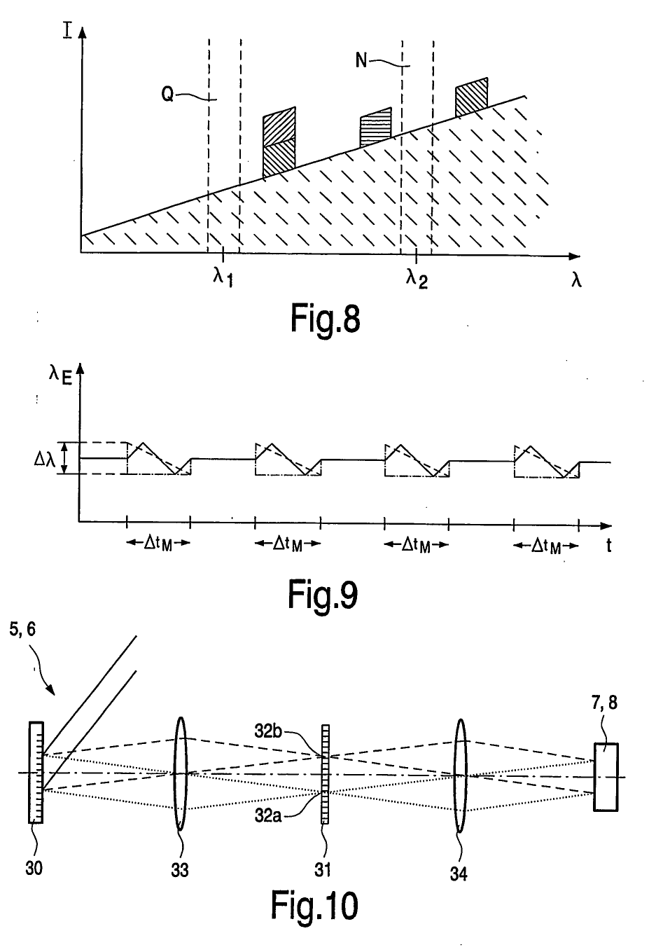

FIG. 8 is a schematic spectrum of the optical signal with modulation of the wavelength of the light provided by the light source;

FIG. 9 is a schematic diagram of the wavelength of the light provided by the light source as function of time;

FIG. 10 is a schematic drawing of a multivariate optical element wherein the weighing function of the multivariate optical element is adjustable and the modulator element is arranged to modulate the weighing function of the multivariate optical element.

The Figures are not drawn to scale. In general, identical components are denoted by the same reference numerals.

In the embodiment shown in FIG. 1 the optical analysis system 20 for determining an amplitude of a principal component of an optical signal comprises a light source 1 for providing light for illuminating a sample 2 comprising a substance having a concentration and thereby generating the principal component. The amplitude of the principal component relates to the concentration of the substance. The light source 1 is a laser such as a gas laser, a dye laser and/or a solid state laser such as a semiconductor or diode laser.

The optical analysis system 20 is part of a blood analysis system 40. The sample 2 comprises skin with blood vessels. The substance may be one or more of the following analytes: glucose, lactate, cholesterol, oxy-hemoglobin and/or desoxy-hemoglobin, glycohemoglobin (HbA1c), hematocrit, cholesterol (total, HDL, LDL), triglycerides, urea, albumin, creatinin, oxygenation, pH, bicarbonate and many others. The concentrations of these, substances is to be determined in a non-invasive way using optical spectroscopy. To this end the light provided by the light source 1 is sent to a dichroic mirror 3 which reflects the light provided by the light source towards the blood vessels in the skin. The light may be focused on-the-blood vessel using an objective 12. The light may be focused in the blood vessel by using an imaging and analysis system as described in the international patent application WO 02/057759.

By interaction of the light provided by the light source 1 with the blood in the blood vessel an optical signal is generated due to Raman scattering and fluorescence. The optical signal thus generated may be collected by the objective 12 and sent to the dichroic mirror 3. The optical signal has a different wavelength than the light provided by the light source 1. The dichroic mirror is constructed such that it transmits at least a portion of the optical signal.

A spectrum of the optical signal generated in this way is shown in FIG. 2A. The spectrum comprises a relatively broad fluorescence background FBG and relatively narrow Raman bands RB. The x-axis of FIG. 2A denotes the wavelength shift with respect to the 785 nm of the excitation by light source 1 in wave numbers, the y-axis of FIG. 2A denotes the intensity in arbitrary units. The x-axis corresponds to zero intensity. The wavelength and the intensity of the Raman bands, i.e. the position and the height, is indicative for the type of analyte as is shown in the example of FIG. 2B for the analyte glucose which was dissolved in a concentration of 80 mMol in water. The solid line of FIG. 2B shows the spectrum of both glucose and water, the dashed line of FIG. 2B shows the difference between the spectrum of glucose in water and the spectrum of water without glucose. The amplitude of the spectrum with these bands is indicative for the concentration of the analyte.

Because blood comprises many compounds each having a certain spectrum which may be as complex as that of FIG. 2B, the analysis of the spectrum of the optical signal is relatively complicated. In the optical analysis system 20 according to the invention the optical signal is analyzed by a multivariate optical element 5, 6 which weighs the optical signal by a weighing function shown schematically in FIGS. 3 and 5. The weighing function of FIG. 3 is designed for glucose in blood. It comprises a positive part P and a negative part N. The positive part P and the negative part N each comprise in this example more than one spectral band. A beam splitter 4 transmits a part of the optical signal to the multivariate optical element 5 which weighs it according to the positive part of the weighing function. This optical signal weighed by the multivariate optical element 5 is detected by detector 7 which is photodiode. Alternatively, any other detector suitable for providing an electrical signal in dependence of the intensity of the weighed optical signal may be used. The beam splitter 4 reflects a part of the optical signal to the multivariate optical element 6 which weighs it according to the negative part of the weighing function. This optical signal weighed by the multivariate optical element 6 is detected by detector 8 which may be identical to the detector 7. A computational element 9 is arranged to calculate the difference between the positive and negative signal. This difference is proportional to the amplitude of the principal component of the optical signal. The amplitude of the principal component relates to the concentration of the substance, i.e. of the analyte. The relation between the amplitude and the concentration may be a linear dependence.

When the principal component comprises only a positive or only a negative component, only one multivariate optical element with one detector may be used.

The multivariate optical element 5, 6 may be any known element having the desired weighing function. Examples of multivariate optical elements are described in U.S. Pat. No. 6,198,531.

The optical signal may comprise in addition to the principal component a further component which is not accounted for when constructing the weighing function. The further component may at least partly be due to dark current of the detector 7 and/or 8, due to background light, which may be induced by the light source 1 by e.g. scattering off optical elements such as the dichroic mirror 3 and/or the objective 12, and/or which may be induced by other sources of light than the light source 1. The further component may at least partly be due to fluorescence of substances which may be other substances than the analytes. For in-vivo blood analysis these substances may include hemoglobin, human skin tissue immersion medium, opticalm elements and/or hygienic cover.

Because the further component is not accounted for in the construction of the weighing function, the weighed optical signal detected by the detector 7, 8 may comprise in addition to a part relating to the amplitude of the principal component a further part relating to a further amplitude of the further component. In other words, the detectors 7, 8 generate an electrical signal from the weighed optical signal which in addition to the amplitude of the principal component depends on the amplitude of the further component.

The presence of a further component is schematically depicted in FIG. 4 which shows the spectrum of the optical signal, i.e. the intensity I in arbitrary units as function of the wavelength λ. In the schematic spectrum of FIG. 4 there are contributions due to compounds A, B and C which were taken into account when designing the spectral-weighing function shown in FIG. 5. The invention is not limited to this number of compounds. According to the invention the number of compounds taken into account may be any positive integer larger than or equal to one. Further there is a contribution and due to background indicated by dashed lines, also referred to as the further component F. The further component was not accounted for when designing the spectral weighing function.

In FIG. 5 the positive part P and the negative part N of the weighing function each comprise one spectral band only. However, the invention is not limited to such relatively simple weighing functions which are chosen here for simplicity of the explanation only. Instead, the spectral weighing function may comprise a positive part with one, two or more spectral bands. The spectral weighing function may further comprise a negative part with one, two or more spectral bands as is shown in FIG. 3.

The intensity of the spectral lines corresponding to the compounds A, B and C relates to the concentration of these compounds in the sample. Often the concentration scales linearly with the intensity of the spectral lines. From the spectrum of FIG. 4 it is clear that the intensity of the spectral line corresponding to compound A is difficult to determine because the spectral line overlaps with one of the spectral lines corresponding to compound B. Alternatively, the spectral lines may partly overlap or may be separated but so close to each other that they cannot be separated by the multivariate optical element because the latter does not have a sufficient spectral accuracy.

In absence of the background one may cope with this difficulty by weighing the optical signal with the spectral weighing function SWF for the principal component corresponding to compound B, shown in FIG. 5. The spectral weighing function may represent the relative transmission or reflection of the multivariate optical element as function of the wavelength. It comprises a positive part P and a negative part N. When there is no further component F, the detected optical signal weighed by the positive part P and the detected optical signal weighed by the negative part N as shown in FIG. 6C are obtained. The difference between the detected optical signal weighed by the positive part P and the detected optical signal weighed by the negative part N is directly proportional to the concentration of the compound B.

When the further component F is present the optical signal weighed by the positive part P and the negative part N are detected as shown in FIG. 6A. For both the negative part and the positive part the detected weighed optical signal comprises a part relating to compounds which were accounted when designing the spectral weighing function, in this example compounds A and B. In addition to this part which relates to the amplitude of the corresponding principal components, the detected weighed optical signal comprises a further part which is due to the further component F. This further part which relates to the amplitude of the further component has an intensity FN in the negative part which is different from the intensity FP in the positive part. As a result the difference between the positive part and the negative part comprises a term relating to the amplitude of the further component and the amplitude of the principal component is determined inaccurately. A similar inaccuracy may also arise when the entire spectral weighing function has the same sign, i.e. when there are no separate positive and negative parts.

To at least partly overcome this inaccuracy the optical analysis system 20 further comprises a modulator element 13 for modulating the detected weighed optical signal such that a difference between the modulated detected weighed optical signal and the detected weighed optical signal relates to the amplitude of the principal component.

In the embodiment of FIG. 1 the modulator element 13 is able to modulate a property of the light provided by the light source 1. The part of the detected weighed optical signal relating to the amplitude of the principal component and the further part of the detected weighed optical signal relating to a further amplitude of the further component depend on the property the light in different ways. Embodiments of this dependence will be given below.

The modulation depth may be 100%, i.e. in the detected weighed optical signal either the part relating to the principal component(s) or to the further component is absent during the modulation. When the detected weighed optical signal either the part relating to the principal component(s) is absent during modulation, the modulated weighed optical signal has the spectrum shown in FIG. 6B. The difference between the modulated detected weighed optical signal and the detected weighed optical signal relating to the amplitude of the principal component is shown in FIG. 6C. It is free from the further component and proportional to the amplitude of the principal component corresponding to compound B. Alternatively, the modulation depth may be smaller than 100%, cf. e.g. the embodiment described below with reference to FIGS. 7A-7D.

The optical analysis system 20 may further comprise a signal processor 9 for determining the amplitude of the principal component from the difference between the modulated detected weighed optical signal and the detected weighed optical signal.

The modulator element 13 may be able to periodically modulate the detected weighed optical signal, the modulation having a frequency and a phase. The signal processor may be able to determine the amplitude of the principal component from the difference between the modulated detected weighed optical signal and the detected weighed optical signal having the frequency and the phase. To this end the modulation by the modulator element 13 may be controlled by a control unit 11 and the signal provided by the signal processor 9 may be processed by a lock-in detector 10 which is well known in the art.

The property of the light to be modulated by the modulator element 13 may be the intensity of the light provided by the light source 1. This modulation may be done by an automated shutter, by a chopper or by modulating an electrical parameter such as the current or the voltage provided to the light source 1. This is e.g. useful when the further component is caused by dark current of the detectors 7, 8 and/or by light originating from a light source other than the light source 1.

Alternatively or in addition, the property of the light to be modulated by the modulator element 13 may comprise the polarization state of the light provided by the light source 1. The modulator element 13 may be a Pockels cell. This modulation scheme may be advantageous when the principal component is at least partly polarized due to e.g. polarized Raman scattering or fluorescence whereas the further component is due to unpolarized light such as unpolarized Raman scattering and/or unpolarized fluorescence. The multivariate optical element may comprise a polarizer which transmits or reflects the part of the optical signal having a predefined polarization state such as linearly or circularly polarized light. The light provided by the light source 1 may have a polarization state such as linearly or circularly polarized light which may be modulated. When modulating the polarization state of the light provided by the light source 1, the part of the detected weighed optical signal relating to the at least polarized principal component is modulated whereas the further part of the detected weighed optical signal relating to the further component is not modulated. This may be used to discriminate these two parts. Alternatively, the principal component may be unpolarized and the further component may be polarized.

In an embodiment the property of the light to be modulated by the modulator element 13 comprises the spectral bandwidth of the light. When a diode laser is used the diode current may be used to set the laser to the multimode domain, e.g. by making use of a relatively broadband optical feedback. Often, the spectrum of the optical signal as shown e.g. in FIG. 4 comprises relatively narrow peaks. The width W of the peak may be determined by relaxation processes specific for the corresponding compound and by the spectral bandwidth of the light generating the optical signal. The spectral shape of the peak may be a convolution of two functions describing; these terms. When the bandwidth of the light generating the optical signal is enlarged during the modulation, the peaks may get a larger width W. When the peaks corresponding to the principal components are smaller than the peak(s) corresponding to the further component as is shown e.g. in FIG. 4 where the peak(s) corresponding to the further component is so broad that it may be referred to as a spectrally varying background rather than as a peak, the former may be changed by the modulation whereas the latter is approximately unchanged. This is depicted in FIG. 7B which shows the spectrum of the optical signal when the spectral bandwidth of the light is modulated. FIG. 7A is the spectrum of the optical signal when the spectral bandwidth of the light is not modulated. It is identical to the spectrum of FIG. 4. In FIG. 7C the spectrum of the modulated detected optical signal weighed by the spectral weighing functions shown in FIG. 5 is shown. It consists mainly of the further component F. The spectrum of the unmodulated detected optical signal weighed by the spectral weighing functions shown in FIG. 5 is shown in FIG. 6A.

The difference between the unmodulated detected optical signal shown in FIG. 4 and the modulated detected optical signal shown in FIG. 7C is shown in FIG. 7D. This difference is approximately equal to but not identical to the one shown in FIG. 6C which depicts the exact principal components. The small differences arise from the fact that small parts relating to compounds A, B and C are detected during modulation as is shown in FIG. 7C. The broader the spectral bandwidth during modulation the smaller this difference is and the larger the modulation depth is.

Instead of modulating the bandwidth itself, the wavelength of the light may be modulated and the modulated signal may be integrated as will be described below with reference to FIG. 9.

In an embodiment the property of the light to be modulated by the modulator element 13 comprises the wavelength of the light. Often, the spectrum of the optical signal as shown e.g. in FIG. 4 comprises relatively narrow peaks each having a central wavelength such as λ₁ and λ₂ shown in e.g. FIG. 4. When the peak is due to Raman scattering the central wavelength of the peak is proportional to the difference of the inverse of the wavelength of the light generating the Raman scattering signal, and the inverse of the wavelength of the vibration involved. When the wavelength of the light generating the Raman scattering signal is changed during the modulation, the spectral position of the corresponding peaks is changed as well. When the further component does not depend on the wavelength of the light generating the optical signal within the tuning range of the wavelength, it is not changed. This may be the case e.g. when the further component is due to fluorescence and when the fluorescence yield is approximately constant over the tuning range. Alternatively or in addition the further component may be due to light originating from light sources others than the light source 1.

In FIG. 8 the spectrum of the optical signal is shown for the example in which the light inducing the optical signal is tuned to a longer wavelength during the modulation. The unmodulated spectrum is the same as in FIG. 4. It is clear from FIGS. 4 and 8 that the further component F is unchanged whereas the position of the peaks corresponding to the compounds A, B and C is shifted to longer wavelength as well. The parts of the spectrum which pass through the positive and the negative multivariate optical element 5 and 6, respectively, having the spectral weighing function shown in FIG. 5 are indicated by dashed lines. The spectrum of the modulated detected optical signal weighed by the spectral weighing functions shown in FIG. 5 is identical to that shown in FIG. 6B. The spectrum of the unmodulated detected optical signal weighed by the spectral weighing functions shown in FIG. 5 is shown in FIG. 6A. The difference between the unmodulated detected optical signal and the modulated detected optical signal is identical to that shown in FIG. 6C. When the change in the wavelength is smaller than the width of the spectral weighing function indicated as V in FIG. 5 the modulation depth is less than 100% and an additional calibration may be required.

The modulated wavelength may have a fixed value during the modulation as is indicated by the dashed dotted line in FIG. 9. Alternatively, the modulated wavelength may vary in time as is indicated as example by solid line and the dashed line in FIG. 9. The detected weighed optical signal may be integrated over the modulation time Δt_{M}. This results in a detected signal which is identical to the one which is obtained in the case of a bandwidth modulation in which the modulated bandwidth is Δλ indicated FIG. 9. This modulation scheme may be useful for optical analysis systems which do not have multivariate optical elements but which may use other means to obtain a spectrum such as e.g. a dispersive element for dispersing the optical signal and detector array such as e.g. a CCD camera for detecting the dispersed optical signal. The dispersed optical signal may be integrated during the modulation to obtain an approximate background. This background may be subtracted from the integrated unmodulated dispersed optical signal. The respective integrations are integration over periods of time and may be implemented in electronics such as an integrator. The difference may be calculated by a signal processor.

In an embodiment of the optical analysis system 20 the modulator element 13 is arranged to directly modulate a property of the optical signal, i.e. it does not modulate the light generating the optical signal but the optical signal itself. In the example of FIG. 1 the modulator element is then located downstream the sample such as e.g. behind the dichroic mirror 3. The property of the optical signal to be modulated by the modulator element 13 may comprise the polarization state of the optical signal. This is analogous to the modulation of the polarization state of the light generating the optical signal described above. The light source 1 may provide polarized light and the optical signal generated thereby may have a polarized principal component and an unpolarized further component. The multivariate optical element 5, 6 may have a polarization dependent transmission and/or reflection. The modulator element 13 may be arranged downstream the sample 2 but upstream the multivariate optical element 5, 6. It may modulate the polarization of the optical signal. The result is that the part of the detected weighed optical signal relating to the amplitude of the principal component and the further part of the detected weighed optical signal relating to a further amplitude of the further component depend on the property the optical signal in different ways. From this difference the amplitude of the principal component may be determined.

The optical analysis system 20 may comprise a multivariate optical element 5, 6 shown e.g. in FIG. 10 wherein the weighing function of the multivariate optical element 5, 6 is adjustable and the modulator element is arranged to modulate the weighing function of the multivariate optical element 5, 6. The multivariate optical element 5, 6 may comprise a dispersive element 30 such as a grating or a prism for spectrally dispersing the optical signal and an weighing element 31 with adjustable segments 32a and 32b for receiving spectral components of the spectrally dispersed optical signal and for distributing the optical signal weighed by the spectral weighing function to the detector 7, 8. In the example of FIG. 10 the weighing element 31 is an array of liquid crystal elements 32 which are sandwiched between two crossed polarizers. The polarizers may be integrated in the liquid crystal elements, e.g. in the substrates confining the liquid crystals. The liquid crystal elements may have an anisotropic index of refraction which for each column is controlled by the voltage V applied to the cells of the column as well known in the art. Depending on the voltage the polarization state of the light incident on the cell may be changed. Due to the change of the polarization state the respective part of the optical signal may be at least partly transmitted and sent to the detector 7, 8. In this way the weighing function may be realized.

In the example of FIG. 10 the multivariate optical element 5, 6 comprises a focusing member 33 which is a lens for focusing the spectrally dispersed optical signal on the weighing element 31, and a further focusing member 34 for re-collimating the weighed optical signal or for focusing it on the detector 7, 8.

The modulator element 13 may be able to modulate the adjustable segments 32. The modulator element 13 may induce a change of the voltage controlling the voltage applied to the liquid crystal cells. In this way the part of the detected weighed optical signal relating to the amplitude of the principal component and the further part of the detected weighed optical signal relating to a further amplitude of the further component depend on the modulated weighing function in different ways.

Instead of an array of liquid crystal elements a transmission filter or a optical density filter with spatially varying transmission may be used which in combination realizes the spectral weighing function. This element may be moved mechanically such that the spectral weighing function is effectively modulated.

In summary, the optical analysis system 20 is arranged to determine an amplitude of a principal component of an optical signal. The optical analysis system 20 comprises a multivariate optical element 5, 6 for weighing the optical signal by a spectral weighing function and a detector 7, 8 for detecting the weighed optical signal. The optical signal comprises the principal component and a further component which was not accounted for when designing the spectral weighing function. Therefore, the detected weighed optical signal comprises a part relating to the amplitude of the principal component and a further part relating to a further amplitude of the further component. The optical analysis system 20 further comprises a modulator element 13 for modulating the detected weighed optical signal. The difference between the modulated detected weighed optical signal and the detected weighed optical signal relates to the amplitude of the principal component and thus allows for determining the amplitude of the principal component in an accurate way. The blood analysis system 40 comprises such an optical analysis system 20. The method of determining an amplitude of an principal component makes use of the optical analysis system 20.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of other elements or steps than those listed in a claim. The word “a” or “an” preceding an element does not exclude the presence of a plurality of such elements.

1. An optical analysis system for determining an amplitude of a principal component of an optical signal, the optical analysis system comprising:
a multivariate optical element for weighing the optical signal by a spectral weighing function, and
a detector for detecting the weighed optical signal,
wherein the optical signal comprises the principal component and a further component, the detected weighed optical signal comprising a part relating to the amplitude of the principal component and a further part relating to a further amplitude of the further component, the optical analysis system further comprising a modulator element for modulating the detected weighed optical signal, wherein a difference between the modulated detected weighed optical signal and the detected weighed optical signal corresponds to the amplitude of the principal component.

2. The optical analysis system as claimed in claim 1, further comprising a signal processor for determining the amplitude of the principal component from the difference between the modulated detected weighed optical signal and the detected weighed optical signal.

3. The optical analysis system as claimed in claim 2, wherein the modulator element modulates the detected weighed optical signal with a frequency and a phase, the signal processor determining the amplitude of the principal component from the difference between the modulated detected weighed optical signal and the detected weighed optical signal having the frequency and the phase.

4. The optical analysis system as claimed in claim 1, further comprising a light source for providing light for illuminating a sample comprising a substance having a concentration and thereby generating the principal component, the amplitude of the principal component relating to the concentration of the substance.

5. The optical analysis system as claimed in claim 4, wherein the modulator element is arranged to modulate a property of the light provided by the light source, the part of the detected weighed optical signal relating to the amplitude of the principal component and the further part of the detected weighed optical signal relating to a further amplitude of the further component depending on the property of the light in different ways.

6. The optical analysis system as claimed in claim 5, wherein the property of the light comprises an intensity of the light.

7. The optical analysis system as claimed in claim 5, wherein the property of the light comprises a polarization state of the light.

8. The optical analysis system as claimed in claim 5, wherein the property of the light comprises a spectral bandwidth of the light.

9. The optical analysis system as claimed in claim 5, wherein the property of the light comprises a wavelength of the light.

10. The optical analysis system as claimed in claim 1, wherein the modulator element is arranged to modulate a property of the optical signal, the part of the detected weighed optical signal relating to the amplitude of the principal component and the further part of the detected weighed optical signal relating to a further amplitude of the further component depending on the property the optical signal in different ways.

11. The optical analysis system as claimed in claim 10, wherein the property of the optical signal comprises a polarization state of the optical signal.

12. The optical analysis system as claimed in claim 1, wherein the weighing function of the multivariate optical element is adjustable and the modulator element is arranged to modulate the weighing function of the multivariate optical element, the part of the detected weighed optical signal relating to the amplitude of the principal component and the further part of the detected weighed optical signal relating to a further amplitude of the further component depending on the modulated weighing function in different ways.

13. The optical analysis system as claimed in claim 12, wherein the multivariate optical element comprises a dispersive element for spectrally dispersing the optical signal and a weighing element with adjustable segments for receiving spectral components of the spectrally dispersed optical signal and for distributing the optical signal weighed by the spectral weighing function to the detector, the modulator element being able to modulate the adjustable segments.

14. (canceled)

15. A method of determining an amplitude of a principal component of an optical signal, the method comprising the steps of:
weighing the optical signal by a multivariate optical element having a spectral weighing function, and
detecting the weighed optical signal by a detector,
wherein the optical signal comprises the principal component and a further component, the detected weighed optical signal comprising a pail relating to the amplitude of the principal component and a further part relating to a further amplitude of the further component, the method further comprising the step of modulating the detected weighed optical signal by a modulator element, wherein a difference between the modulated detected weighed optical signal and the detected weighed optical signal corresponds to the amplitude of the principal component.

16. The method as claimed in claim 15, further comprising the step of calculating the amplitude of the principal component from the modulated weighed optical signal and the unmodulated weighed optical signal.

17. The method as claimed in claim 15 further comprising providing a light source for illuminating a sample comprising a substance having a concentration and thereby generating the principal component, the amplitude of the principal component relating to the concentration of the substance, and modulating a property of light with the modulator element, wherein the part of the detected weighed optical signal relating to the amplitude of the principal component and the further part of the detected weighed optical signal relating to a further amplitude of the further component depending on the property of the light in different ways.

18. The method as claimed in claim 15, wherein the property of the light is one of an intensity of the light, a polarization of the light, a spectral bandwidth of the light, and a wavelength of the light.

19. A blood analysis system comprising:
a multivariate optical element for weighing an optical signal by a spectral weighing function; and
a detector for detecting the weighed optical signal;
wherein the optical signal comprises a principal component and a further component, the detected weighed optical signal comprising a part relating to the amplitude of the principal component and a further part relating to a further amplitude of the further component, the optical analysis system further comprising a modulator element for modulating the detected weighed optical signal, wherein a difference between the modulated detected weighed optical signal and the detected weighed optical signal corresponds to the amplitude of the principal component.

20. The blood analysis system as claimed in claim 19, further comprising a light source for illuminating a sample comprising a substance having a concentration and thereby generating the principal component, the amplitude of the principal component relating to the concentration of the substance, and wherein the modulator element modulates a property of light, wherein the part of the detected weighed optical signal relating to the amplitude of the principal component and the further part of the detected weighed optical signal relating to a further amplitude of the further component depending on the property of the light in different ways.

21. The blood analysis system as claimed in claim 19, wherein the property of the light is one of an intensity of the light, a polarization of the light, a spectral bandwidth of the light, and a wavelength of the light.
